(19) United States
(12) Patent Application Publication
(10) Pub. No.: US 2026/0041675 A1
(43) Pub. Date: Feb. 12, 2026

(54) NASAL COMPOSITION

(71) Applicant: SATO PHARMACEUTICAL CO., LTD., Tokyo (JP)

(72) Inventor: Yoshitoshi KOYAMA, Tokyo (JP)

(73) Assignee: SATO PHARMACEUTICAL CO., LTD., Tokyo (JP)

(21) Appl. No.: 19/239,326

(22) Filed: Jun. 16, 2025

Related U.S. Application Data
(62) Division of application No. 18/638,204, filed on Apr. 17, 2024.
Application No. 18/638,204 is a continuation of application No. PCT/JP2022/022273, filed on Jun. 1, 2022.

(51) Int. Cl. A61K 31/4402; A61K 9/00; A61K 31/4174; A61P 11/02

(52) U.S. Cl. CPC A61K 31/4402; A61K 9/0043; A61K 31/4174; A61P 11/02

(57) ABSTRACT

The purpose of the present invention is to provide: a composition for treating acute rhinitis and allergic rhinitis; and a method for treating acute rhinitis and allergic rhinitis. Specifically, the present invention pertains to a composition that comprises, per 100 mL of the composition, chlorpheniramine or a pharmaceutically acceptable salt thereof in an amount of 200-1000 mg as expressed in terms of chlorpheniramine maleate, and oxymetazoline or a pharmaceutically acceptable salt thereof in an amount of 10-100 mg as expressed in terms of oxymetazoline hydrochloride; and a method for treating acute rhinitis and allergic rhinitis by spraying the composition into a nostril once a day or twice a per day at an interval of 10 hours or more.

### CROSS-REFERENCE TO RELATED APPLICATIONS

This application is a Divisional of U.S. patent application Ser. No. 18/638,204 filed Apr. 17, 2024, which is a Continuation of International Application No. PCT/JP2022/022273 filed Jun. 1, 2022, the entire contents of which are incorporated herein by reference.

The pharmaceutical composition disclosed herein has been marketed in Japan since Sep. 13, 2021 under the trademark of Nasivin® Medi with approval by the PMDA (Pharmaceuticals and Medical Devices Agency) of Japan.

### TECHNICAL FIELD

The present invention relates to a composition for treating acute rhinitis and allergic rhinitis, use thereof, and a method for treating acute rhinitis and allergic rhinitis.

### BACKGROUND ART

Acute rhinitis and allergic rhinitis are diseases of the nasal mucosa, whose major symptoms include paroxysmal or repetitive sneezing, rhinorrhea, and nasal blockage. Among these major symptoms, nasal blockage often afflicts patients with secondary symptoms such as neurological symptoms, sleep disturbance, mouth breathing, and occupational mental disorders.

Acute rhinitis and allergic rhinitis are caused by various causative substances. Acute rhinitis is caused by, for example, viruses, bacteria, dust, chemicals, air pollutants, and the like. Allergic rhinitis is caused by, for example, pollen and house dust, such as mites, mold, and human dander, and hair of pets, e.g., dogs and cats. In some patients, not only a single substance but also two or more substances are often the cause. Even if the causative substance is the same, symptoms vary from person to person.

Acute rhinitis and allergic rhinitis caused by such a wide variety of causes not only affect many patients, but also tend to increase in recent years.

Acute rhinitis and allergic rhinitis are difficult to completely cure, and the main treatment thereof is symptomatic therapy. As drugs for that purpose, a wide variety of drugs have been developed, such as steroids, vasoconstrictors, antihistamines, chemical mediator release inhibitors, α-sympathomimetics, anti-leukotrienes, anti-prostaglandin D2-thromboxane A2 drugs, Th2 cytokine inhibitors, and crude drugs, and new drugs are still being developed.

However, acute rhinitis and allergic rhinitis are caused by a wide variety of substances. The efficacy of a single drug is therefore limited.

For this reason, nasal compositions containing multiple active ingredients have been developed. This aims to achieve superior effects that cannot be achieved with a single active ingredient. In particular, there is a demand for the development of a combination that is highly effective, long lasting, and safe for patients with symptoms of sneezing and/or rhinorrhea in addition to nasal blockage.

However, it is difficult even for those skilled in the art to predict what effects are specifically achieved by which combinations of known active ingredients. In addition, the effect of a nasal composition formed by combining multiple active ingredients is not automatically determined by the combination of active ingredients per se, but varies depending on the ratio and amount of each component blended, and also changes depending on the administration thereof. It is extremely difficult even for those skilled in the art to predict this change, and it is not known unless it is confirmed by animal experiments and clinical trials.

Moreover, as described above, a wide variety of active ingredients are known as therapeutic agents for acute rhinitis and allergic rhinitis, so that the number of possible candidate combinations is per se enormous. It can be said that it is a matter of great interest to those skilled in the art what kind of therapeutic effect will be achieved when changing the dosage, administration, and the like for each of this enormous number of combinations. However, having an interest and actually conducting animal experiments and clinical trials to confirm the effects thereof are completely different issues. This is because animal experiments and clinical trials require a great deal of time and money. There are too many combinations, dosages, administrations, and the like that those skilled in the art are interested in and would like to confirm in future animal experiments and clinical trials, but have not yet been implemented.

Under the above circumstances, there is Respibien (registered trademark) as a nasal composition containing a combination of chlorpheniramine maleate and oxymetazoline hydrochloride as active ingredients. Respibien contains 50 mg/100 mL of chlorpheniramine maleate and 50 mg/100 mL of oxymetazoline hydrochloride.

Respibien is a nasal composition approved for production and sale by the Spanish medical administrative authority, the Spanish Agency of Medicines and Medical Devices (AEMPS), and the agency publishes technical data for Respibien in its online pharmaceutical information service, CIMA. The technical data states that the efficacy of Respibien is “local and temporary relief of the nasal blockage of allergic rhinitis” (Section 4.1). It also states that its administration is “up to 3 consecutive days in the absence of better medical advice” (Section 4.4 Special Warnings and Precautions for Use).

There is no doubt that Respibien has been confirmed, by animal tests and clinical trials, to be effective and safe when it is administered as stated in the technical data, and approved for production and sale by the Spanish Agency of Medicines and Medical Devices. As far as nasal compositions containing a combination of chlorpheniramine maleate and oxymetazoline hydrochloride are concerned, the technical data is the only evidence confirmed by animal tests and clinical trials, prior to the present invention. There is no other substantiated data other than the above. The statements described in the technical data of Respibien, which state it is for “local and temporary relief of the nasal blockage of allergic rhinitis” and that its administration is “up to 3 consecutive days,” are the only efficacy and administration supported by animal tests and clinical trials. It can therefore be rightly said that the efficacy and the administration are common general technical knowledge for those skilled in the art.

Patent specifications sometimes disclose examples referred to as paper examples or prophetic examples. These are clearly distinguished from working examples disclosing tests and experiments that were actually carried out in technical fields where the effects based on the configuration of the invention are poorly predictable, in particular, in the fields of medicament and medicine. The existence of patent specifications with paper examples or prophetic examples is no exception for nasal compositions as well. For example, U.S. Pat. No. 6,824,762 (and its patent family documents), Japanese Patent Application Publication No. 2005-75735, Japanese Patent Application Publication No. 2006-131627, Japanese Patent Application Publication No. 2006-151955, and Japanese Patent Application Publication No. 2006-213700 disclose examples of such types of nasal compositions that contain various combinations of active ingredients. Among them are nasal compositions that contain the same combination of active ingredients as that of Respibien, but in different amounts.

However, these patent documents do not specifically disclose the effects and the like of the nasal compositions of the examples.

### SUMMARY OF INVENTION

### Problems to be Solved by the Invention

An object of the present invention is to provide a composition for treating acute rhinitis and allergic rhinitis, use thereof, and a method for treating acute rhinitis and allergic rhinitis using the same.

The present invention particularly aims to provide a nasal composition for use in the treatment of patients with acute rhinitis and allergic rhinitis who have developed sneezing and/or rhinorrhea in addition to nasal blockage, use thereof, and a method for treating acute rhinitis and allergic rhinitis using the same.

The present invention aims to provide a nasal composition for use in the treatment of patients with acute rhinitis and allergic rhinitis who have developed sneezing and/or rhinorrhea in addition to nasal blockage by nasal drops once or twice a day, use thereof, and a method for treating acute rhinitis and allergic rhinitis using the same.

The present invention further aims to provide a highly effective and safe nasal composition for use in the treatment of patients with acute rhinitis and allergic rhinitis who have developed sneezing and/or rhinorrhea in addition to nasal blockage, use thereof, and a method for treating acute rhinitis and allergic rhinitis using the same.

### Means for Solution of the Problems

Surprisingly, it has been found that a composition containing specific amounts of chlorpheniramine maleate and oxymetazoline hydrochloride as active ingredients can be highly effective, long-lasting, and safe for patients of acute rhinitis and allergic rhinitis who have developed sneezing and/or rhinorrhea in addition to nasal blockage.

The composition of the present invention contains 200 to 1000 mg, preferably 200 to 600 mg, more preferably 400 to 600 mg, of chlorpheniramine in terms of chlorpheniramine maleate, and 10 to 100 mg, preferably 45 to 50 mg, of oxymetazoline in terms of oxymetazoline hydrochloride, per 100 mL of the composition. The composition of the present invention most preferably contains 500 mg of chlorpheniramine maleate and 50 mg of oxymetazoline hydrochloride per 100 mL of the composition.

### Advantageous Effects of the Invention

When administered once or twice a day at intervals of 10 hours or longer, the composition of the present invention safely relieves, with high efficacy and persistence, the major symptoms of patients with acute rhinitis and allergic rhinitis who have developed sneezing and/or rhinorrhea in addition to nasal blockage.

### BRIEF DESCRIPTION OF DRAWINGS

FIG. 1A shows the variation in the mean value of scores found for the swelling of the concha nasalis inferior mucosa on day 1 of administration of the composition of the present invention and on the day of final visit, as diagnosed by the investigators.

FIG. 1B shows the variation in the mean value of scores found for the color tone of the concha nasalis inferior mucosa on day 1 of administration of the composition of the present invention and on the day of final visit, as diagnosed by the investigators.

FIG. 1C shows the variation in the mean value of scores found for the watery secretion levels on day 1 of administration of the composition of the present invention and on the day of final visit, as diagnosed by the investigators.

FIG. 1D shows the variation in the mean value of scores found for the characteristics of rhinorrhea on day 1 of administration of the composition of the present invention and on the day of final visit, as diagnosed by the investigators.

FIG. 2A shows the variation in the mean value of subjective symptom scores for paroxysmal sneezing by the subjects from day 2 to day 6 of administration, based on the day before the start of administration.

FIG. 2B shows the variation in the mean value of subjective symptom scores for nasal discharge by the subjects from day 2 to day 6 of administration, based on the day before the start of administration.

FIG. 2C shows the variation in the mean value of subjective symptom scores for nasal blockage by the subjects from day 2 to day 6 of administration, based on the day before the start of administration.

FIG. 2D shows the variation in the mean value of subjective symptom scores for the three nasal symptoms by the subjects from day 2 to day 6 of administration, based on the day before the start of administration.

FIG. 2E shows the variation in the mean value of subjective symptom scores for the troubles with daily life by the subjects from day 2 to day 6 of administration, based on the day before the start of administration.

### DESCRIPTION OF EMBODIMENTS

The present inventor has conducted research on nasal compositions that can be long-lasting and highly safe, with a fewer number of administration, for patients of acute rhinitis and allergic rhinitis who have developed sneezing and/or rhinorrhea in addition to nasal blockage. As a result, it has been found surprisingly that nasal compositions containing specific amounts of chlorpheniramine maleate and oxymetazoline hydrochloride, when administered once or twice a day at intervals of 10 hours or more, are capable of providing a long-lasting and safe alleviation for patients who have developed such symptoms.

The present invention includes the followings:
1) A method of treating acute rhinitis and/or allergic rhinitis; comprising a step of spraying a composition to a nostril of a patient with acute rhinitis and/or allergic rhinitis once or twice a day, at intervals of 10 hours or longer, for a period of up to 7 days;
   wherein the composition comprises (a) chlorpheniramine or its pharmaceutically acceptable salt and (b) oxymetazoline or its pharmaceutically acceptable salt; and
   wherein the amount of (a) in terms of chlorpheniramine maleate is 200 to 1000 mg per 100 mL of the composition and the amount of (b) in terms of oxymetazoline hydrochloride is 10 to 100 mg per 100 mL of the composition.
2) The method according to claim 1, wherein the amount of (a) is 200 to 600 mg per 100 mL of the composition.
3) The method according to claim 2, wherein the amount of (b) is 45 to 100 mg per 100 mL of the composition.
4) The method according to claim 3, wherein the amount of (a) is 400 to 600 mg per 100 mL of the composition.
5) The method according to claim 4, wherein the amount of (b) is 45 to 50 mg per 100 mL of the composition.
6) The method according to claim 5, wherein the amount of (a) is 400 to 500 mg per 100 ml of the composition.
7) The method according to claim 6, wherein the amount of (a) is 500 mg per 100 mL of the composition.
8) The method according to claim 6, wherein the amount of (b) is 50 mg per 100 mL of the composition.
9) The method according to claim 1, wherein (a) is chlorpheniramine maleate, and (b) is oxymetazoline hydrochloride.
10) The method according to claim 1, wherein the patient has developed symptoms of acute rhinitis and/or allergic rhinitis.
11) A composition comprising (a) chlorpheniramine or its pharmaceutically acceptable salt and (b) oxymetazoline or its pharmaceutically acceptable salt;
   wherein the amount of (a) in terms of chlorpheniramine maleate is 200 to 1000 mg per 100 mL of the composition and the amount of (b) in terms of oxymetazoline hydrochloride is 10 to 100 mg per 100 mL of the composition; and
   wherein the composition is sprayed to a nostril of a patient with acute rhinitis and/or allergic rhinitis once or twice a day, at intervals of 10 hours or longer, for a period of up to 7 days to treat acute rhinitis and/or allergic rhinitis.
12) The composition according to claim 11, wherein the amount of (a) is 200 to 600 mg per 100 mL of the composition.
13) The composition according to claim 12, wherein the amount of (b) is 45 to 100 mg per 100 mL of the composition.
14) The composition according to claim 13, wherein the amount of (a) is 400 to 600 mg per 100 mL of the composition.
15) The composition according to claim 14, wherein the amount of (b) is 45 to 50 mg per 100 mL of the composition.
16) The composition according to claim 15, wherein the amount of (a) is 400 to 500 mg per 100 mL of the composition.
17) The composition according to claim 16, wherein the amount of (a) is 500 mg per 100 mL of the composition.
18) The composition according to claim 16, wherein the amount of (b) is 50 mg per 100 mL of the composition.
19) The composition according to claim 11, wherein (a) is chlorpheniramine maleate, and (b) is oxymetazoline hydrochloride.

The nasal composition of the present invention contains chlorpheniramine or a pharmaceutically acceptable salt thereof. It is known that chlorpheniramine is a first-generation histamine receptor antagonist and suppresses inflammation and airway secretions by competitively antagonizing Hi receptors.

The nasal composition of the present invention contains 200 to 1000 mg of chlorpheniramine or a pharmaceutically acceptable salt thereof in terms of chlorpheniramine maleate per 100 mL of the composition. The amount of chlorpheniramine or a pharmaceutically acceptable salt thereof blended is preferably 400 to 600 mg, more preferably 500 mg, in terms of chlorpheniramine maleate per 100 mL of the composition.

When the nasal composition of the present invention contains a chlorpheniramine-free form, conversion to chlorpheniramine maleate can be carried out as follows. When the content of the chlorpheniramine-free form in the composition is denoted by Xc, the amount converted to chlorpheniramine maleate is denoted by Yc, the molecular weight of the chlorpheniramine-free form is denoted by Ac, and the molecular weight of chlorpheniramine maleate is denoted by Bc, Yc is given by the following formula (I).

$$Yc = Xc \times Bc / Ac \qquad formula ( I )$$

Similarly, when the nasal composition contains chlorpheniramine mesylate, the conversion to chlorpheniramine maleate can be calculated according to the above formula (I), with the chlorpheniramine mesylate content in the composition as Xc and the molecular weight of chlorpheniramine mesylate as Ac.

The nasal composition of the present invention contains oxymetazoline or a pharmaceutically acceptable salt thereof. It is known that oxymetazoline belongs to imidazoline-based compounds, and is a vasoconstrictor having a sympathomimetic action.

The nasal composition of the present invention contains 10 to 100 mg of oxymetazoline or a pharmaceutically acceptable salt thereof in terms of oxymetazoline hydrochloride per 100 mL of the composition. The amount of oxymetazoline or a pharmaceutically acceptable salt thereof blended is preferably 40 to 50 mg, more preferably 50 mg, in terms of oxymetazoline hydrochloride per 100 mL of the composition.

When the nasal composition contains free body of oxymetazoline, the conversion to oxymetazoline hydrochloride can be made as follows. When the content of the free body of oxymetazoline in the composition is Xo, the amount converted to oxymetazoline hydrochloride is Yo, the molecular weight of the free body of oxymetazoline is Ao, and the molecular weight of oxymetazoline hydrochloride is Bo, Yo can be given by the following formula (II).

$${Y}_{o} = {X}_{o} \times {B}_{o} / {A}_{o} \qquad formula ( II )$$

Similarly, when the nasal composition of the present invention contains oxymetazoline mesylate, conversion to oxymetazoline hydrochloride can be calculated according to the above formula (II), with the oxymetazoline mesylate content in the composition as Xo and the molecular weight of oxymetazoline mesylate as Ao.

The numerical range described above are exemplary. The nasal spray composition of the present invention can contain the above-described chlorpheniramine or salt thereof, oxymetazoline or salt thereof, in an appropriate combination within the numerical ranges described above.

The chlorpheniramine salt blended in the nasal composition of the present invention is not particularly limited as long as it is pharmaceutically acceptable, and examples thereof include the maleate, hydrochloride, hydrobromide, mesylate, phosphate, sulfate, and tartrate salts of chlorpheniramine, with chlorpheniramine maleate being preferred. The chlorpheniramine salt may be either the dl-form or the d-form, and when the d-form is used, the amount is usually ½ of the dl-form.

The oxymetazoline salt blended in the nasal composition of the present invention is not particularly limited as long as it is pharmaceutically acceptable, and examples thereof include the hydrochloride, hydrobromide, maleate, mesylate, sulfate, and tartrate salts of oxymetazoline, with oxymetazoline hydrochloride being preferred.

In addition to chlorpheniramine or a salt thereof and oxymetazoline or a salt thereof, the nasal composition of the present invention can contain ingredients effective for the treatment of acute rhinitis or allergic rhinitis, such as steroids, chemical mediator release inhibitors, α-sympathomimetics, anti-leukotrienes, anti-prostaglandin D₂·thromboxane A₂ drugs, and Th₂ cytokine inhibitors, as long as the effects of the present invention are not impaired.

The nasal composition of the present invention may contain a bactericide or preservative, such as benzalkonium chloride, a p-hydroxybenzoic acid ester, Hibiten, benzethonium chloride, benzalkonium chloride, chlorhexidine hydrochloride, or chlorhexidine gluconate. Among these, benzalkonium chloride is preferred.

The nasal composition of the present invention may contain an isotonic agent such as sodium chloride or glycerine. Among these, sodium chloride is preferred.

The nasal composition of the present invention can contain a chelating agent such as edetic acid, ethylenediaminediacetic acid, citric acid, tartaric acid, succinic acid, or a salt thereof. Among these, an edetate is preferred.

The nasal composition of the present invention may contain a buffering agent such as sodium chloride, sodium monohydrogen phosphate, potassium monohydrogen phosphate, sodium dihydrogen phosphate, potassium dihydrogen phosphate, or boric acid. Among these, potassium monohydrogen phosphate and potassium dihydrogen phosphate are preferred.

The nasal composition of the present invention is adjusted to a pH of 5.5 to 6.5, preferably 6.0, with a pH adjuster.

In addition to the above, the nasal composition of the present invention may contain items commonly used in nasal compositions, including thickeners such as polyvinyl alcohol and polyacrylic acid; anti-inflammatory agents such as dipotassium glycyrrhizinate, methyl salicylate, acetaminophen, and indomethacin; secretion inhibitors such as belladonna total alkaloids, and enzyme anti-inflammatory agents such as lysozyme chloride; local anesthetics such as lidocaine hydrochloride, benzocaine, procaine hydrochloride, and ethyl aminobenzoate; vitamins such as vitamin A and vitamin C; and refreshing agents such as menthol and eucalyptus oil, as long as the effects of the present invention are not impaired.

The nasal composition of the present invention is sprayed into the nostrils once or twice a day. Each spray is at intervals of 10 hours or longer.

Spraying can be carried out using, for example, a 30 to 70 μg, preferably 40 to 60 μg metered dose inhaler. When using a metered-dose inhaler of 50 μg, for example, each nasal cavity should be sprayed 2-3 times per dose.

The safety of administration has been confirmed up to 7 days. Once symptoms subside, administration can be terminated at that time.

The nasal composition of the present invention can be produced by mixing chlorpheniramine or a salt thereof, oxymetazoline or a salt thereof, and the optional ingredients described above and formulating them into a desired dosage form. The nasal composition of the present invention can be formulated into sprays, liniments, and the like, and sprays are preferred.

### EXAMPLES

Hereinafter, the present invention is described with reference to examples. The examples are intended to illustrate the present invention, not to limit the present invention.

### Example 1 (Animal Experimentation 1)

The following experiment was carried out using 2,4-toluene diisocyanate (TDI)-sensitized guinea pigs, a model of nasal hypersensitivity.

### Sensitization:

After quarantine and acclimatization of 4-week-old male Hartley strain guinea pigs, a 10% TDI ethyl acetate solution, soaked in an otolaryngological swab, was applied by contact to the bilateral nasal vestibules of the guinea pigs for 10 seconds. This operation was carried out once a day for 5 consecutive days. Twenty-four days after the last application, the same operation was repeated once a day for 5 consecutive days.

### Induction of Sneezing Response:

Nine days after the end of sensitization, a 5% TDI ethyl acetate solution, soaked in an otolaryngological swab, was applied by contact to the bilateral nasal vestibules of the guinea pigs for 10 seconds to induce a sneezing response.

### Calculation of Symptom Score:

Symptoms were observed for 10 minutes after the sneezing reaction was induced, and the symptoms were evaluated according to the evaluation items and scores shown in Table 1, and the symptom score was calculated according to the following formula.

$$Symptom score = score for sneezing + score for nose scratch count + score for watery rhinorrhea + score for nasal blockage sound .$$

TABLE 1
Evaluation items and scores

| | Score | | | |
|---|---|---|---|---|
| | 0 | 1 | 2 | 3 |
| Sneezing | None | 1 to 4 | 5 to 11 | 12 Times |
| | | Times | Times | or More |
| Nose Scratch | 0 Times | 1 to 19 | 20 to 39 | 40 Times |
| Count | | Times | Times | or More |
| Watery | None | Visible at | Between 1 | Drips out |
| rhinorrhea | | entrance | and 3 | of nose |
| | | of nose | | |
| Nasal Blockage | None | Yes | — | — |
| Sound | | | | |

The above sneeze induction and symptom score evaluations were performed once a day for each animal for a total of 3 days (3 cycles in total), and the mean value of the symptom scores for 3 cycles was calculated.

### Selection and Grouping of Guinea Pigs:

In the next week following the end of induction, guinea pigs for which the mean value of symptom scores for 3 cycles was less than 8 and guinea pigs with wheezing were excluded. The selected guinea pigs were grouped into groups A to D (10 animals in each group) by the stratified randomization method using the mean value of symptom scores for 3 cycles for each individual as an index.

### Test Items:

The following test items A to D were administered to the guinea pigs of each group from the day after grouping.
A: Placebo
B: Composition containing 500 mg of chlorpheniramine maleate and 50 mg of oxymetazoline hydrochloride in 100 mL of the composition
C: Composition containing 500 mg of chlorpheniramine maleate in 100 mL of the composition
D: Composition containing 50 mg of oxymetazoline hydrochloride in 100 mL of the composition

All the above A to D contain the following amounts of additives and residual water per 100 mL.

| | | |
|---|---|---|
| Benzalkonium Chloride | 10 | mg |
| Sodium Chloride | 550 | mg |
| Disodium Edetate Hydrate | 50 | mg |
| Potassium Dihydrogen Phosphate | 500 | mg |

| | |
|---|---|
| Dipotassium hydrogenphosphate | Amount to |
| | make pH = 6 |

### Administration of Test Items:

A piston pipette was used to nasally administer 200 μL to the bilateral nasal cavities of guinea pigs (100 μL per nasal cavity on one side). Thirty minutes after the nasal administration, a 5% TDI ethyl acetate solution, soaked in an otolaryngological swab, was applied by contact to the bilateral nasal vestibules of each guinea pig for 10 seconds to induce a sneezing response. The symptoms were observed for 10 minutes after the induction, and the calculation method shown in the section “Calculation of symptom score” above was used to calculate the mean value of symptom scores for each animal. Thus, the results in Table 2 (mean value and standard error) were obtained.

TABLE 2

| | Amount of | Concentration of Active Ingredient | | Mean Value and |
|---|---|---|---|---|
| | Solution | Chlorpheniramine | Oxymetazoline | Standard Error of |
| Group | Administered | Maleate | Hydrochloride | Symptom Scores |
| A | 200 μL | — | — | 8.5 ± 0.3 |
| B | 200 μL | 0.5% (500 mg)* | 0.05% (50 mg)* | 4.4 ± 0.5 |
| C | 200 μL | 0.5% (500 mg)* | — | 6.1 ± 0.6 |
| D | 200 μL | — | 0.05% (50 mg)* | 7.1 ± 0.5 |

*Content of each component per 100 mL of test items

### Example 2 (Clinical Trial)

With the cooperation of 4 medical institutions, a phase III open-label general clinical trial was carried out on a nasal composition containing 0.5% chlorpheniramine maleate and 0.05% oxymetazoline hydrochloride as active ingredients, and containing the same additives in the same amounts, as in test substance B of Example 1.

### Selection of Subjects:

Patients who met the following conditions were included in the clinical trial.
1) Patients aged 15 years or older at the time of informed consent. For patients under the age of 20, their legally authorized representative with decision-making capacity must provide consent.
2) Gender is irrelevant.
3) Outpatients at the 4 medical institutions were included, while their inpatients were excluded because they were more likely to have serious complications.
4) The severity of each nasal symptom (paroxysmal sneezing, rhinorrhea, and nasal blockage) was determined according to Table 3 below, “Evaluation criteria for the severity of each nasal symptom”, and patients whose severity does not fall under “Most Severe” in Table 4 below were chosen as subject candidates. This is because patients who have the severity++++ for any of nasal blockage, rhinorrhea, and sneezing are likely to be judged by the investigators to require concomitant use with other therapies.

TABLE 3
Evaluation criteria for the severity of each nasal symptom

| | Severity | | | | |
|---|---|---|---|---|---|
| | ++++ | +++ | ++ | + | − |
| Type | (4 Points) | (3 Points) | (2 Points) | (1 Point) | (0 Points) |
| Paroxysmal Sneezing | 21 Times | 20 to 11 | 10 to 6 | 5 to 1 | Below+ |
| (Number of Attacks | or More | Times | Times | Times | |
| per Day) | | | | | |
| Rhinorrhea | 21 Times | 20 to 11 | 10 to 6 | 5 to 1 | Below+ |
| (Number of Nose- | or More | Times | Times | Times | |
| Blowing per Day) | | | | | |
| Nasal Blockage | Completely | Severe nasal | Severe nasal | Nasal | Below+ |
| | obstructed | blockage | blockage | blockage | |
| | all day | causing | causing | without | |
| | | prolonged | occasional | oral | |
| | | oral | oral | breathing | |
| | | breathing in | breathing in | | |
| | | a day | a day | | |

TABLE 4
Classification of the severity of allergy rhinitis symptom

| | | Paroxysmal Sneezing or Rhinorrhea* | | | |
|---|---|---|---|---|---|
| Degree** and Severity | | ++++ | +++ | ++ | + |
| Nasal | ++++ | Most | Most | Most | Most |
| Blockage | | Severe | Severe | Severe | Severe |
| | +++ | Most | Severe | Severe | Severe |
| | | Severe | | | |
| | ++ | Most | Severe | Moderate | Moderate |
| | | Severe | | | |
| | + | Most | Severe | Moderate | Mild |
| | | Severe | | | |

*Select more severe one, sneezing or rhinorrhea
**The degree of each nasal symptom is determined according to Table 3. For nasal blockage, the severity of the most severe symptoms in the morning, noon, and night is used.

5) Patients with acute rhinitis who are estimated to be within 2 days after onset, have a body temperature of 37.5° C. or lower, and do not exhibit symptoms of sore throat, cough, or phlegm.
6) Patients with allergic rhinitis who are positive (class or score 2 or more) in CAP-RAST or RAST (allergy-causing antigen test).

However, in consideration of subject safety, efficacy of the clinical trial, and the like, patients who fall under any of the following items are excluded from the targets of the present clinical trial at the investigator's discretion.
a) Patients with complicated nasal disease that affects the evaluation of nasal symptoms.
b) Patients on hyposensitization therapy or non-specific immunotherapy.
c) Patients who have undergone concha nasalis inferior mucosal resection, submucosal inferior turbinate resection, etc., within 5 years.
d) Patients who cannot avoid receiving concomitant drugs or therapy that may affect the clinical trial.
e) Patients who received any concomitant drug or therapy affecting the clinical trial within 14 days (28 days for systemic steroids) before the start of administering the investigational product.
f) Patients with coronary artery disease, hypertension, hyperthyroidism, diabetes, or glaucoma.
g) Patients with severe respiratory disease and chronic respiratory disease.
h) Patients with liver disease, renal disease, heart disease, asthma, or recurrent epistaxis.
i) Patients with or history of drug-induced hypersensitivity.
j) Pregnant patients and lactating patients.
k) Patients who have participated in other clinical trials within the past 6 months.
l) Other patients who are judged to be inappropriate by the investigators.

Sixty-nine people, who met the above criteria 1) to 4) and 5) or 6) and were not excluded by a) to 1), were selected as subjects for the present clinical trial. The subjects were diagnosed and broken down into 10 with acute rhinitis, 33 with perennial allergic rhinitis, 14 with seasonal allergic rhinitis+perennial allergic rhinitis, and 12 with acute rhinitis+allergic rhinitis. The subjects with allergic rhinitis had a mean disease duration of 17.0 years.

### Overview of Clinical Trial

A nasal composition containing 50 mg of oxymetazoline hydrochloride and 500 mg of chlorpheniramine maleate in 100 mL is sprayed into each nasal cavity 2 or 3 times at a time, 1 or 2 times per day, using a 50 g metered dose inhaler. The interval between applications should be 10 to 12 hours or longer. The subjects continue to follow this administration and dosage until they receive a doctor's examination at the final visit on day 7 after the initial day of administration, in principle. At that point, any metered dose inhaler containing residual medicament is collected.

### Therapeutic Effect Evaluation 1 (Observation by Investigators)

Examination by the investigators before the start of administration: The investigators examined the subjects who visited the hospital on the initial day of administration, and determined the nasal cavity findings (swelling of the concha nasalis inferior mucosa, color tone of the concha nasalis inferior mucosa, watery secretion level, and characteristics of rhinorrhea) according to Table 5.

TABLE 5
Criteria for the severity of nasal cavity findings (objective symptoms)

| | Severity | | | |
|---|---|---|---|---|
| | +++ | ++ | + | − |
| Type | (3 Points) | (2 Points) | (1 Point) | (0 Points) |
| Swelling of Concha | Middle nasal | Between (+++) | Center of | No swelling |
| Nasalis Inferior | concha could | and (+) | middle nasal | |
| Mucosa | not be seen | | concha could | |
| | | | be seen | |
| Color Tone of Concha | Pale | Red | Pale Red | Normal |
| Nasalis Inferior | | | | |
| Mucosa* | | | | |
| Watery Secretion Level | Filled | Between (+++) | Adhered | No Secretion |
| | | and (+) | | |
| Characteristics of | Watery | Viscous | Purulent | No Rhinorrhea |
| Rhinorrhea* | | | | |

*Not severity but quality

After the above examination, the subjects received the first nasal administration according to the administration described in the aforementioned overview of clinical trial. After that, nasal administration was continued according to the same administration.

Among the 69 subjects, 66 had a final visit on day 7 as originally scheduled, 2 had a final visit on day 6, and 1 had a final visit on day 8. It was confirmed by patient diaries and interviews on the day of final visit to be described later that all 69 subjects complied with the above administration and that they did not use other medicaments or therapy during the administration period.

### Changes in Nasal Cavity Findings (Objective Symptoms):

The investigators made a diagnosis of the subjects' nasal cavity findings (objective symptoms), conducted on the initial day of administration according to Table 5, also on the day of final visit. The diagnosis results on both days were scored with 3 points for +++, 2 points for ++, 1 point for +, and 0 points for −, and the mean values for the 69 subjects were calculated as shown in Table 6 below.

TABLE 6
Changes in the scores of nasal cavity findings
(objective symptoms) by the investigators

| | | | Mean | | Max | | Min |
|---|---|---|---|---|---|---|---|
| Item | | | Value | S.D. | Value | Median | Value |
| Swelling | Measured | Initial Day of | 2.0 | 0.6 | 3 | 2.0 | 0 |
| of Concha | Value | Administration | | | | | |
| Nasalis | | Day of Final | 1.1 | 0.6 | 3 | 1.0 | 0 |
| Inferior | | Visit | | | | | |
| Mucosa | Variation | Day of Final | −0.9 | 0.7 | 1 | −1.0 | −2 |
| | | Visit | | | | | |
| Color Tone | Measured | Initial Day of | 2.0 | 0.7 | 3 | 2.0 | 1 |
| of Concha | Value | Administration | | | | | |
| Nasalis | | Day of Final | 1.6 | 0.9 | 3 | 1.0 | 0 |
| Inferior | | Visit | | | | | |
| Mucosa | Variation | Day of Final | −0.5 | 1.0 | 1 | 0.0 | −3 |
| | | Visit | | | | | |
| Watery | Measured | Initial Day of | 1.7 | 0.5 | 3 | 2.0 | 0 |
| Secretion | Value | Administration | | | | | |
| Level | | Day of Final | 1.0 | 0.6 | 2 | 1.0 | 0 |
| | | Visit | | | | | |
| | Variation | Day of Final | −0.7 | 0.7 | 1 | −1.0 | −2 |
| | | Visit | | | | | |
| Characteristics | Measured | Initial Day of | 2.7 | 0.7 | 3 | 3.0 | 0 |
| of Rhinorrhea | Value | Administration | | | | | |
| | | Day of Final | 2.2 | 1.3 | 3 | 3.0 | 0 |
| | | Visit | | | | | |
| | Variation | Day of Final | −0.5 | 1.1 | 1 | 0.0 | −3 |
| | | Visit | | | | | |

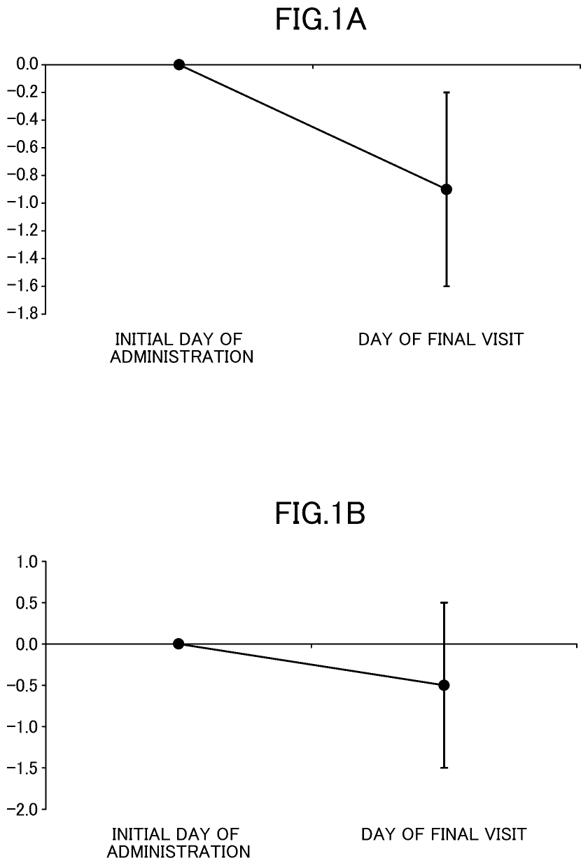

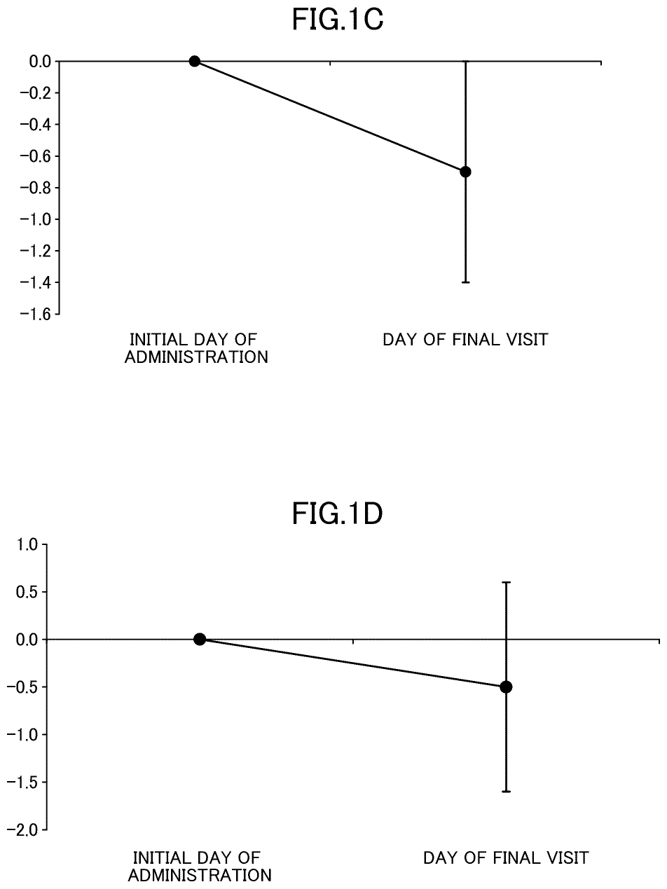

The mean values for nasal cavity findings (objective symptoms) shown in Table 6 are illustrated in FIGS. 1A to 1D. It is apparent from FIGS. 1A to 1D that judging from the mean scores for nasal cavity findings (objective symptoms) by the investigators, the swelling of the concha nasalis inferior mucosa, the color tone of the concha nasalis inferior mucosa, the watery secretion level, and the characteristics of rhinorrhea all improved on the day of final visit compared to the initial day of administration.

Table 7 shows in more detail the changes in nasal cavity findings (objective symptoms) by the investigators on the initial day of administration and the day of final visit. Regarding Table 7, to explain Swelling of Concha Nasalis Inferior Mucosa taken as an example item, the upper rows for the item indicate finding scores according to Table 5 above, “Criteria for the severity of nasal cavity findings (objective symptoms)”, and the lower rows indicate improvement step counts of the finding scores.

TABLE 7
Changes in nasal cavity finding scores (count values)

| | Initial Day of | Day of Final Visit | | | |
|---|---|---|---|---|---|
| Item | Administration | 3 | 2 | 1 | 0 |
| Swelling of | 3 | 1 | 2 | 7 | 0 |
| Concha Nasalis | 2 | 0 | 11 | 33 | 5 |
| Inferior Mucosa | 1 | 0 | 1 | 4 | 4 |
| | 0 | 0 | 0 | 0 | 1 |
| | | | Number | | |
| | | | of Cases | (%) | |
| | Improved 3 Steps | | 0 | (0.0) | |
| | Improved 2 Steps | | 12 | (17.4) | |
| | Improved 1 Step | | 39 | (56.5) | |
| | Unchanged | | 17 | (24.6) | |
| | Worsened 1 Step | | 1 | (1.4) | |
| | Worsened 2 Steps | | 0 | (0.0) | |
| | Worsened 3 Steps | | 0 | (0.0) | |
| Color Tone of | 3 | 9 | 1 | 8 | 2 |
| Concha Nasalis | 2 | 6 | 13 | 11 | 2 |
| Inferior Mucosa | 1 | 0 | 2 | 13 | 2 |
| | 0 | 0 | 0 | 0 | 0 |
| | | | Number | | |
| | | | of Cases | (%) | |
| | Improved 3 Steps | | 2 | (2.9) | |
| | Improved 2 Steps | | 10 | (14.5) | |
| | Improved 1 Step | | 14 | (20.3) | |
| | Unchanged | | 35 | (50.7) | |
| | Worsened 1 Step | | 8 | (11.6) | |
| | Worsened 2 Steps | | 0 | (0.0) | |
| | Worsened 3 Steps | | 0 | (0.0) | |
| Watery | 3 | 0 | 0 | 1 | 0 |
| Secretion | 2 | 0 | 11 | 31 | 7 |
| Level | 1 | 0 | 3 | 10 | 4 |
| | 0 | 0 | 0 | 0 | 2 |
| | | | Number | | |
| | | | of Cases | (%) | |
| | Improved 3 Steps | | 0 | (0.0) | |
| | Improved 2 Steps | | 8 | (11.6) | |
| | Improved 1 Step | | 35 | (50.7) | |
| | Unchanged | | 23 | (33.3) | |
| | Worsened 1 Step | | 3 | (4.3) | |
| | Worsened 2 Steps | | 0 | (0.0) | |
| | Worsened 3 Steps | | 0 | (0.0) | |
| Characteristics | 3 | 42 | 2 | 0 | 9 |
| of Rhinorrhea | 2 | 5 | 3 | 1 | 4 |
| | 1 | 0 | 0 | 0 | 0 |
| | 0 | 0 | 0 | 0 | 3 |
| | | | Number | | |
| | | | of Cases | (%) | |
| | Improved 3 Steps | | 9 | (13.0) | |
| | Improved 2 Steps | | 4 | (5.8) | |
| | Improved 1 Step | | 3 | (4.3) | |
| | Unchanged | | 48 | (69.6) | |
| | Worsened 1 Step | | 5 | (7.2) | |
| | Worsened 2 Steps | | 0 | (0.0) | |
| | Worsened 3 Steps | | 0 | (0.0) | |

The results in Table 7 above were evaluated according to the determination table, Table 8 below, and the total values for “Markedly Improved” or better and “Improved” or better are shown in Table 9 below.

TABLE 8
Improvement determination table for nasal cavity findings

| Score and Degree | | Day of Final Visit | | | |
|---|---|---|---|---|---|
| of Improvement | | 3.0 | 2.0 | 1.0 | 0 |
| Initial Day of | 3.0 | Unchanged | Improved | Markedly | Disappeared |
| Administration | | | | Improved | |
| | 2.0 | Worsened | Unchanged | Improved | Disappeared |
| | 1.0 | Worsened | Worsened | Unchanged | Disappeared |
| | 0 | Worsened | Worsened | Worsened | No Relevant |
| | | | | | Findings* |

*Case where the findings score is 0 (zero) in both the diagnosis on day 1 of administration and the day of final visit

TABLE 9
Degree of improvement in nasal cavity findings (day of final visit)

| | Number of Cases (Percentage (%)**) | | | | | | | Improvement Rate (%) | |
|---|---|---|---|---|---|---|---|---|---|
| | | | | | | No | | 95% Confidence Interval | |
| | | Markedly | | | | Relevant | | “Markedly Improved” | “Improved” |
| Item | Disappeared | Improved | Improved | Unchanged | Worsened | Findings | Total* | or Better | or Better |
| Swelling of | 9 | 7 | 35 | 16 | 1 | 1 | 68 | 23.5 | 75.0 |
| Concha Nasalis | (13.2) | (10.3) | (51.5) | (23.5) | (1.5) | | | 14.1 to 35.4 | 63.0.to 84.7 |
| Inferior Mucosa | | | | | | | | | |
| Color Tone of | 6 | 8 | 12 | 35 | 8 | 0 | 69 | 20.3 | 37.7 |
| Concha Nasalis | (8.7) | (11.6) | (17.4) | (50.7) | (11.6) | | | 11.6 to 31.7 | 26.3 to 50.2 |
| Inferior Mucosa | | | | | | | | | |
| Watery secretion | 11 | 1 | 31 | 21 | 3 | 2 | 67 | 17.9 | 64.2 |
| level | (16.4) | (1.5) | (46.3) | (31.3) | (4.5) | | | 9.6 to 29.2 | 51.5 to 75.5 |
| Characteristics | 13 | 0 | 3 | 45 | 5 | 3 | 66 | 19.7 | 24.2 |
| of rhinorrhea | (19.7) | (0.0) | (4.5) | (68.2) | (7.6) | | | 10.9 to 31.3 | 14.5 to 36.4 |

*Number of cases except for “No Relevant Findings”
**Percentage calculated by excluding the cases of “No Relevant Findings”

As shown in Table 9, the improvement rate of nasal cavity findings on the day of final visit was clearly improved for all items.

### Therapeutic Effect Evaluation 2 (Subjective Evaluation by Subjects)

Prior to the aforementioned diagnosis on day 1 of administration by the investigators, the subjects received a patient's diary and an explanation, and consented to fill in the following i) to iv) in the patient's diary.
i) Self-assessment of nasal symptoms (paroxysmal sneezing, rhinorrhea, nasal blockage, and troubles with daily life) according to the criteria in Table 10 below and entry into the patient's diary. The date of entry shall be from the day before the first visit to the day before the day of final visit, excluding day 1 of administration and the day of the last visit, when the subjects are examined by the investigators. Three symptoms of nasal blockage, rhinorrhea, and sneezing are entered three times in the morning, noon, and night.
ii) The subject's impressions of own nasal symptoms and impressions of the sustained effects of the present investigational product should also be entered in the patient's diary.
iii) The number of nasal administrations of the present clinical trial composition shall be entered on each day from day 1 of administration until returning the metered dose nasal dropper containing the present clinical trial composition on the day of final visit.
iv) Body temperature shall be measured and recorded on each day from the initial day of administration to the day of final visit, and if the body temperature exceeds 37.5° C., they shall contact with the investigators.

TABLE 10
Evaluation criteria for the severity of each nasal symptom

| | Severity | | | | |
|---|---|---|---|---|---|
| | ++++ | +++ | ++ | + | − |
| Type | (4 Points) | (3 Points) | (2 Points) | (1 Point) | (0 Points) |
| Paroxysmal | 21 Times | 20 to 11 | 10 to 6 | 5 to 1 | Less Than+ |
| Sneezing | or More | Times | Times | Times | |
| (Number of | | | | | |
| Attacks per Day) | | | | | |
| Rhinorrhea | 21 Times | 20 to 11 | 10 to 6 | 5 to 1 | Less Than+ |
| (Number of Nose- | or More | Times | Times | Times | |
| Blowing per Day) | | | | | |
| Nasal Blockage | Completely | Severe nasal | Serious nasal | Without mouth | Less Than+ |
| | stuffed | blockage, and | blockage, and | breathing but | |
| | all day | mouth breathing | mouth breathing | with nasal | |
| | | for a great many | for occasional | blockage | |
| | | hours of the day | hours of the day | | |
| Troubles with | Completely | Unbearably | Between (+++) | Not interfering | Less Than+ |
| daily life* | impossible | painful | and (+) | much | |

*Troubles with daily life: Difficulty in work, study, housework, sleep, going out, and the like

The results of self-evaluation of paroxysmal sneezing, rhinorrhea, nasal blockage, and troubles with daily life by the subjects according to the criteria in Table 10 were as in Table 11 below. In Table 11, the upper rows for each item indicate the symptoms and the like for the item, scored with 4 points for ++++, 3 points for +++, 2 points for ++, 1 point for +, and 0 points for −, and the lower rows indicate improvement step counts of the finding scores.

TABLE 11-1
Changes in nasal symptom scores (subjective symptoms by the subjects)

| | Day before | Day 2 of | | | | | Day 3 of | | | | | Day 4 of | | | | | Day 5 of | | | | | Day 6 of | | | | |
|---|---|---|---|---|---|---|---|---|---|---|---|---|---|---|---|---|---|---|---|---|---|---|---|---|---|---|
| | Initial Day of | Administration | | | | | Administration | | | | | Administration | | | | | Administration | | | | | Administration | | | | |
| Item | Administration | 4 | 3 | 2 | 1 | 0 | 4 | 3 | 2 | 1 | 0 | 4 | 3 | 2 | 1 | 0 | 4 | 3 | 2 | 1 | 0 | 4 | 3 | 2 | 1 | 0 |
| Proxysmal | 3 | 1 | 9 | 5 | 10 | 3 | 1 | 6 | 10 | 10 | 1 | 1 | 6 | 8 | 11 | 2 | 2 | 3 | 8 | 13 | 2 | 0 | 5 | 10 | 12 | 0 |
| Sneezing | 2 | 0 | 2 | 10 | 9 | 2 | 0 | 0 | 11 | 11 | 1 | 0 | 2 | 8 | 13 | 0 | 0 | 1 | 6 | 15 | 1 | 0 | 0 | 7 | 14 | 2 |
| | 1 | 0 | 0 | 2 | 15 | 1 | 0 | 0 | 1 | 17 | 0 | 0 | 0 | 1 | 15 | 2 | 0 | 0 | 0 | 15 | 3 | 0 | 0 | 0 | 13 | 4 |
| | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| | Total | 1 | 11 | 17 | 34 | 6 | 1 | 6 | 22 | 38 | 2 | 1 | 8 | 17 | 39 | 4 | 2 | 4 | 14 | 43 | 6 | 0 | 5 | 17 | 39 | 6 |
| | | Number of Cases (%) | | | | | Number of Cases (%) | | | | | Number of Cases (%) | | | | | Number of Cases (%) | | | | | Number of Cases (%) | | | | |
| | Improved by | 3 (4.3) | | | | | 1 (1.4) | | | | | 2 (2.9) | | | | | 2 (2.9) | | | | | 0 (0.0) | | | | |
| | 3 Steps | | | | | | | | | | | | | | | | | | | | | | | | | |
| | Improved by | 12 (17.4) | | | | | 11 (15.9) | | | | | 11 (15.9) | | | | | 14 (20.3) | | | | | 14 (20.9) | | | | |
| | 2 Steps | | | | | | | | | | | | | | | | | | | | | | | | | |
| | Improved by | 15 (21.7) | | | | | 21 (30.4) | | | | | 23 (33.3) | | | | | 26 (37.7) | | | | | 28 (41.8) | | | | |
| | 1 Step | | | | | | | | | | | | | | | | | | | | | | | | | |
| | Unchanged | 34 (49.3) | | | | | 34 (49.3) | | | | | 29 (42.0) | | | | | 24 (34.8) | | | | | 25 (37.3) | | | | |
| | Worsened by | 5 (7.2) | | | | | 2 (2.9) | | | | | 4 (5.8) | | | | | 3 (4.3) | | | | | 0 (0.0) | | | | |
| | 1 Step | | | | | | | | | | | | | | | | | | | | | | | | | |
| | Worsened by | 0 (0.0) | | | | | 0 (0.0) | | | | | 0 (0.0) | | | | | 0 (0.0) | | | | | 0 (0.0) | | | | |
| | 2 Steps | | | | | | | | | | | | | | | | | | | | | | | | | |
| | Worsened by | 0 (0.0) | | | | | 0 (0.0) | | | | | 0 (0.0) | | | | | 0 (0.0) | | | | | 0 (0.0) | | | | |
| | 3 Steps | | | | | | | | | | | | | | | | | | | | | | | | | |
| Rhinorrhea | 3 | 2 | 11 | 14 | 7 | 1 | 1 | 10 | 13 | 11 | 0 | 1 | 12 | 8 | 12 | 2 | 2 | 6 | 15 | 12 | 0 | 2 | 7 | 13 | 9 | 3 |
| (Runny | 2 | 0 | 3 | 14 | 3 | 0 | 0 | 4 | 11 | 5 | 0 | 0 | 2 | 10 | 8 | 0 | 0 | 0 | 12 | 8 | 0 | 0 | 0 | 10 | 9 | 1 |
| | 1 | 1 | 0 | 1 | 9 | 2 | 1 | 0 | 2 | 9 | 1 | 0 | 2 | 0 | 10 | 1 | 0 | 1 | 1 | 10 | 1 | 0 | 0 | 2 | 10 | 0 |
| | 0 | 0 | 0 | 0 | 1 | 0 | 0 | 0 | 0 | 1 | 0 | 0 | 0 | 0 | 1 | 0 | 0 | 0 | 0 | 1 | 0 | 0 | 0 | 0 | 1 | 0 |
| | Total | 3 | 14 | 29 | 20 | 3 | 2 | 14 | 26 | 26 | 1 | 1 | 16 | 18 | 31 | 3 | 2 | 7 | 28 | 31 | 1 | 2 | 7 | 25 | 29 | 4 |

| | Number of Cases (%) | Number of Cases (%) | Number of Cases (%) | Number of Cases (%) | Number of Cases (%) |
|---|---|---|---|---|---|
| Improved by | 1 (1.4) | 0 (0.0) | 2 (2.9) | 0 (0.0) | 3 (4.5) |
| 3 Steps | | | | | |
| Improved by | 7 (10.1) | 11 (15.9) | 12 (17.4) | 12 (17.4) | 10 (14.9) |
| 2 Steps | | | | | |
| Improved by | 19 (27.5) | 19 (27.5) | 17 (24.6) | 24 (34.8) | 22 (32.8) |
| 1 Step | | | | | |
| Unchanged | 34 (49.3) | 30 (43.5) | 32 (46.4) | 28 (40.6) | 27 (40.3) |
| Worsened by | 7 (10.1) | 8 (11.6) | 4 (5.8) | 4 (5.8) | 5 (7.5) |
| 1 Step | | | | | |
| Worsened by | 0 (0.0) | 0 (0.0) | 2 (2.9) | 1 (1.4) | 0 (0.0) |
| 2 Steps | | | | | |
| Worsened by | 1 (1.4) | 1 (1.4) | 0 (0.0) | 0 (0.0) | 0 (0.0) |
| 3 Steps | | | | | |

TABLE 12
Changes in nasal symptom scores (subjective symptoms by the subjects and measured values)

| | | | Measured Value | | | | | Variation | | | | |
|---|---|---|---|---|---|---|---|---|---|---|---|---|
| | | Number | Avg. | | Max | | Min | Avg. | | Max | | Min. |
| Item | | of Cases | Value | S.D. | Value | Median | Value | Value | S.D. | Value | Median | Value |
| Paroxysmal | Day Before | 69 | 2.1 | 0.8 | 3 | 2.0 | 1 | — | — | — | — | — |
| Sneezing | Start of | | | | | | | | | | | |
| | Administration | | | | | | | | | | | |
| | Day 2 of | 69 | 1.5 | 0.9 | 4 | 1.0 | 0 | −0.6 | 1.0 | 1 | 0.0 | −3 |
| | Administration | | | | | | | | | | | |
| | Day 3 of | 69 | 1.5 | 0.8 | 4 | 1.0 | 0 | −0.6 | 0.8 | 1 | 0.0 | −3 |
| | Administration | | | | | | | | | | | |
| | Day 4 of | 69 | 1.5 | 0.8 | 4 | 1.0 | 0 | −0.7 | 0.9 | 1 | −1.0 | −3 |
| | Administration | | | | | | | | | | | |
| | Day 5 of | 69 | 1.3 | 0.8 | 4 | 1.0 | 0 | −0.8 | 0.9 | 1 | −1.0 | −3 |
| | Administration | | | | | | | | | | | |
| | Day 6 of | 67 | 1.3 | 0.7 | 3 | 1.0 | 0 | −0.8 | 0.8 | 0 | −1.0 | −2 |
| | Administration | | | | | | | | | | | |
| Rhinorhea | Day Before | 69 | 2.3 | 0.8 | 3 | 3.0 | 0 | — | — | — | — | — |
| (Runny | Start of | | | | | | | | | | | |
| Nose) | Administration | | | | | | | | | | | |
| | Day 2 of | 69 | 1.9 | 0.9 | 4 | 2.0 | 0 | −0.4 | 1.0 | 3 | 0.0 | −3 |
| | Administration | | | | | | | | | | | |
| | Day 3 of | 69 | 1.9 | 0.9 | 4 | 2.0 | 0 | −0.4 | 1.0 | 3 | 0.0 | −2 |
| | Administration | | | | | | | | | | | |
| | Day 4 of | 69 | 1.7 | 0.9 | 4 | 2.0 | 0 | −0.6 | 1.0 | 2 | 0.0 | −3 |
| | Administration | | | | | | | | | | | |
| | Day 5 of | 69 | 1.7 | 0.8 | 4 | 2.0 | 0 | −0.6 | 0.9 | 2 | −1.0 | −2 |
| | Administration | | | | | | | | | | | |
| | Day 6 of | 67 | 1.6 | 0.9 | 4 | 2.0 | 0 | −0.7 | 1.0 | 1 | −1.0 | −3 |
| | Administration | | | | | | | | | | | |
| Nasal | Day Before | 69 | 2.2 | 0.7 | 3 | 2.0 | 1 | — | — | — | — | — |
| Blockage | Start of | | | | | | | | | | | |
| (Stuffy | Administration | | | | | | | | | | | |
| Nose) | Day 2 of | 69 | 1.7 | 1.0 | 4 | 2.0 | 0 | −0.4 | 0.9 | 2 | 0.0 | −2 |
| | Administration | | | | | | | | | | | |
| | Day 3 of | 69 | 1.6 | 0.8 | 4 | 2.0 | 0 | −0.5 | 0.9 | 2 | 0.0 | −2 |
| | Administration | | | | | | | | | | | |
| | Day 3 of | 69 | 1.4 | 0.7 | 3 | 1.0 | 0 | −0.8 | 0.9 | 2 | −1.0 | −3 |
| | Administration | | | | | | | | | | | |
| | Day 5 of | 69 | 1.3 | 0.8 | 3 | 1.0 | 0 | −0.9 | 0.9 | 1 | −1.0 | −2 |
| | Administration. | | | | | | | | | | | |
| | Day 6 of | 67 | 1.2 | 0.8 | 3 | 1.0 | 0 | −0.9 | 1.0 | 1 | −1.0 | −3 |
| | Administration | | | | | | | | | | | |
| Troubles | Day Before | 69 | 1.3 | 0.7 | 3 | 1.0 | 0 | — | — | — | — | — |
| with | Start of | | | | | | | | | | | |
| Daily | Administration | | | | | | | | | | | |
| Life | Day 2 of | 69 | 0.9 | 0.7 | 3 | 1.0 | 0 | −0.4 | 0.8 | 1 | 0.0 | −3 |
| | Administration | | | | | | | | | | | |
| | Day 3 of | 69 | 0.9 | 0.7 | 3 | 1.0 | 0 | −0.4 | 0.9 | 1 | 0.0 | −3 |
| | Administration | | | | | | | | | | | |
| | Day 4 of | 69 | 0.8 | 0.7 | 3 | 1.0 | 0 | −0.5 | 0.9 | 2 | 0.0 | −3 |
| | Administration | | | | | | | | | | | |
| | Day 5 of | 69 | 0.8 | 0.7 | 3 | 1.0 | 0 | −0.6 | 0.8 | 2 | −1.0 | −2 |
| | Adiministration | | | | | | | | | | | |
| | Day 6 of | 67 | 0.7 | 0.7 | 3 | 1.0 | 0 | −0.6 | 0.8 | 1 | −1.0 | −2 |
| | Administration | | | | | | | | | | | |
| Mean | Day Before | 69 | 2.20 | 0.58 | 3.0 | 2.30 | 1.0 | — | — | — | — | — |
| Score of | Start of | | | | | | | | | | | |
| Three | Administration | | | | | | | | | | | |
| Nasal | Day 2 of | 69 | 1.72 | 0.75 | 4.0 | 1.70 | 0.0 | −0.48 | 0.78 | 1.6 | −0.30 | −2.4 |
| Symptoms | Administration | | | | | | | | | | | |
| | Day 3 of | 65 | 1.67 | 0.65 | 3.7 | 1.70 | 0.3 | −0.53 | 0.72 | 1.0 | −0.30 | −2.0 |
| | Administration | | | | | | | | | | | |
| | Day 4 of | 69 | 1.52 | 0.68 | 3.3 | 1.30 | 0.3 | −0.68 | 0.79 | 1.3 | −0.60 | −2.7 |
| | Administration | | | | | | | | | | | |
| | Day 5 of | 69 | 1.43 | 0.65 | 3.7 | 1.30 | 0.3 | −0.77 | 0.72 | 1.0 | −0.70 | −2.0 |
| | Administration | | | | | | | | | | | |
| | Day 6 of | 67 | 1.39 | 0.64 | 3.3 | 1.30 | 0.0 | −0.82 | 0.70 | 0.6 | −0.70 | −2.7 |
| | Administration | | | | | | | | | | | |

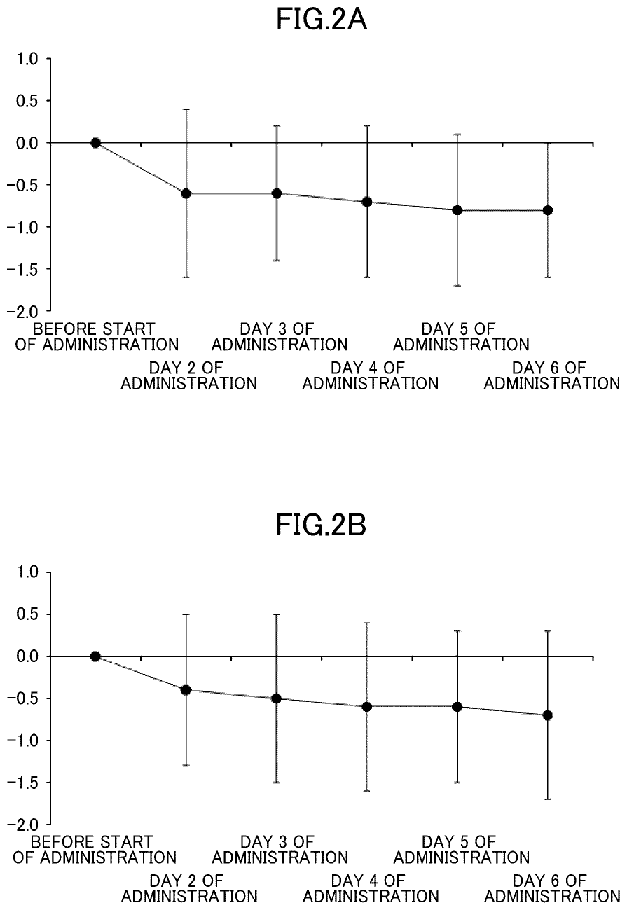

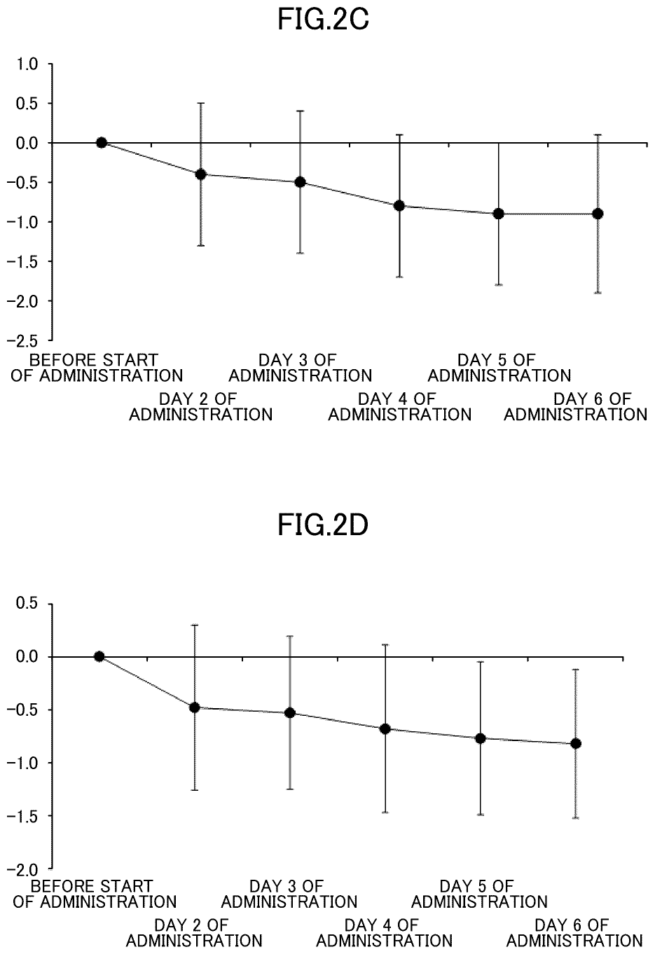

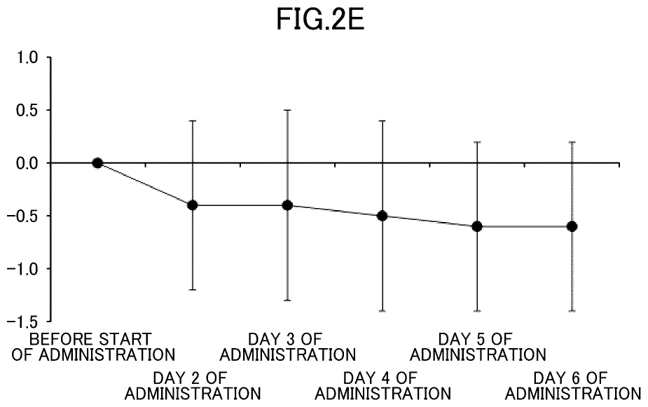

For each of the paroxysmal sneezing, rhinorrhea, and nasal blockage, the mean value of subject scores on the day before the initial day of administration and from day 2 of administration to the day before the day of final visit were calculated (see Table 12), and when the mean value on the day before the initial day of administration is used as a reference (=0) to plot their variations, the results are as shown in FIGS. 2A to 2C. Similar plotting of the changes in mean values of the three nasal symptoms (paroxysmal sneezing, rhinorrhea, and nasal blockage) yields FIG. 2D, and similar plotting of changes in the troubles with daily life yields FIG. 2E. Here, the vertical lines at day 2 of administration to day 6 of administration indicate the standard deviation.

Each graph clearly shows that the subjective symptoms by the subjects were continuously improved until day 6 of administration.

For the degrees of improvement in paroxysmal sneezing, rhinorrhea, nasal blockage, and degree of disability in daily life shown in Table 11 above, evaluation was made according to the criteria shown in Table 13 on the intermediate day of administration (day 3 of administration) and the day before the final visit.

TABLE 13
Determination table for the degree of improvement in nasal symptoms

| | | Intermediate Day (Day 3 of Administration) and Day | | | | |
|---|---|---|---|---|---|---|
| | | before Day of Final Visit (Day 6 of Administration) | | | | |
| Score and Degree | | | 3.0 or More | 2.0 or More | 1.0 or More | |
| of Improvement | | 4.0 or More | Less Than 4.0 | Less Than 3.0 | Less Than 2.0 | Less Than 1.0 |
| Day before | 3.0 or More | Worsened | Unchanged | Improved | Markedly | Disappeared |
| Initial Day of | Less Than 4.0 | | | | Improved | |
| Administration | 2.0 or More | Worsened | Worsened | Unchanged | Improved | Disappeared |
| | Less Than 3.0 | | | | | |
| | 1.0 or More | Worsened | Worsened | Worsened | Unchanged | Disappeared |
| | Less Than 2.0 | | | | | |
| | Less Than 1.0 | Worsened | Worsened | Worsened | Worsened | No Relevant |
| | | | | | | Symptom* |

*Case where the subjective score for the relevant symptom is 0 (zero) on both the intermediate day and the day before the final visit

TABLE 14
Degree of improvement in each nasal symptom (paroxysmal sneezing, rhinorrhea, and nasal blockage) and troubles with daily life

| | | | | | | | | | Improvement Rate (%) | |
|---|---|---|---|---|---|---|---|---|---|---|
| | | Number of Cases (Percentage (%)**) | | | | | | | 95% Confidence Interval | |
| | | | | | | | No | | “Markedly | |
| | Day of | | Markedly | | | | Relevant | | Improved” | “Improved” |
| Item | Evaluation | Disappeared | Improved | Improved | Unchanged | Worsened | Symptom | Total* | or Better | or Better |
| Paroxysmal | Intermediate | 2 | 10 | 21 | 34 | 2 | 0 | 69 | 17.4 | 47.8 |
| Sneezing | Day | (2.9) | (14.5) | (30.4) | (49.3) | (2.9) | | | 9.3 to 28.4 | 35.6 to 60.2 |
| | Day before | 6 | 13 | 24 | 26 | 0 | 0 | 69 | 27.5 | 62.3 |
| | Day of Final | (8.7) | (18.8) | (34.8) | (37.7) | (0.0) | | | 17.5 to 39.6 | 49.8 to 73.7 |
| | Visit | | | | | | | | | |
| Rhinorrhea | Intermediate | 1 | 11 | 18 | 30 | 9 | 0 | 69 | 17.4 | 43.5 |
| (Runny | Day | (1.4) | (15.9) | (26.1) | (43.5) | (13.0) | | | 9.3 to 28.4 | 31.6 to 56.0 |
| Nose) | Day before | 4 | 9 | 23 | 28 | 5 | 0 | 69 | 18.8 | 52.2 |
| | Day of Final | (5.8) | (13.0) | (33.3) | (40.6) | (7.2) | | | 10.4 to 30.1 | 39.8 to 64.4 |
| Nasal | Intermediate | 6 | 6 | 22 | 28 | 7 | 0 | 69 | 17.4 | 49.3 |
| Blockage | Day | (8.7) | (8.7) | (31.9) | (40.6) | (10.1) | | | 9.3 to 28.4 | 37.0 to 61.6 |
| (Stuffy | Day before | 9 | 12 | 26 | 17 | 5 | 0 | 69 | 30.4 | 68.1 |
| Nose) | Day of Final | (13.0) | (17.4) | (37.7) | (24.6) | (7.2) | | | 19.9 to 42.7 | 55.8 to 78.8 |
| | Visit | | | | | | | | | |
| Troubles | Intermediate | 18 | 1 | 11 | 28 | 9 | 2 | 67 | 28.4 | 44.8 |
| with Daily | Day | (26.9) | (1.3) | (16.4) | (41.8) | (13.4) | | | 18.0 to 40.7 | 32.6 to 57 4 |
| Life | Day before | 25 | 2 | 12 | 24 | 3 | 3 | 66 | 40.9 | 59.1 |
| | Day of Final | (37.9) | (3.0) | (18.2) | (36.4) | (4.3) | | | 29.0 to 53.7 | 46.3 to 71.0 |
| | Visit | | | | | | | | | |

*Except for “No Relevant Symptom”
**Percentage calculated by excluding the cases of “No Relevant Symptom”

It is apparent from Table 14 that for all of the items of paroxysmal sneezing, rhinorrhea, nasal blockage, and troubles with daily life, the improvement of therapeutic effects progressed significantly from day 3 of administration to the day before the day of final visit.

### Subject's Impressions of Nasal Symptoms:

In the present clinical trial, subjects were asked to evaluate their impressions of nasal symptoms on the following 5-point scale and record them in the patient's diary.
1: Much improved
2: Improved
3: Somewhat improved
4: No change
5: Worsened

Among the impressions of nasal symptoms recorded in the patient's diaries by 69 subjects, evaluations on day 3 of administration and the day before the day of final visit were as shown in Table 15 below.

TABLE 15
Subject's impressions (nasal symptoms)

| | | | | | | | Impression Rate (%) | |
|---|---|---|---|---|---|---|---|---|
| | | | | | | | 95% Confidence Interval | |
| | Number of Cases (Percentage (%)) | | | | | | | “Somewhat |
| Day of | Much | | Somewhat | No | | | “Improved” | Improved” |
| Evaluation | Improved | Improved | Improved | Change | Worsened | Total | or Better | or Better” |
| Day 3 of | 8 | 11 | 27 | 22 | 1 | 69 | 27.5 | 66.7 |
| Administration | (11.6) | (15.9) | (39.1) | (31.9) | (1.4) | | 17.5 to 39.6 | 54.3 to 77.6 |
| Day before | 11 | 25 | 24 | 8 | 1 | 69 | 52.2 | 87.0 |
| Day of Final | (15.9) | (36.2) | (34.8) | (11.6) | (1.4) | | 39.8 to 64.4 | 76.7 to 93.9 |
| Visit | | | | | | | | |

As shown in Table 15, the improvement rate in the subject's impressions of nasal symptoms reached 27.5% for “Improved” or better and 66.7% for “Somewhat Improved” or better on day 3 of administration, and further increased to 52.2% for “Improved” or better and 87.0% for “Somewhat Improved” or better on the day before the day of final visit.

### Subject's Impressions of Sustained Effects:

present clinical trial, the impressions of the sustained effects of the present investigational product on sneezing and runny nose and the sustained effects on nasal blockage were evaluated on the following 4-point scale and asked to be recorded in the patient's diary.
1: Satisfied, 2: Somewhat satisfied, 3: Somewhat dissatisfied, 4: Dissatisfied

For the impressions of these sustained effects recorded by the subjects in the patient's diaries, the evaluations on day 3 of administration and the day before the day of final visit were as shown in Table 16.

TABLE 16
Subject's impressions (sustained effects)

| | | | | | | | | Satisfaction Rate (%) | |
|---|---|---|---|---|---|---|---|---|---|
| | | | | | | | | 95% Confidence Interval | |
| | | | Number of Cases (Percentage (%)) | | | | | | “Somewhat |
| | | Day of | | Somewhat | Somewhat | | | | Satisfied” |
| Item | | Evaluation | Satisfied | Satisfied | Dissatisfied | Dissatisfied | Total | “Satisfied” | or Better |
| Sustained | Paroxysmal | Intermediate | 17 | 31 | 19 | 2 | 59 | 24.6 | 69.6 |
| Effects | Sneezing and | Day | (24.6) | (44.9) | (27.5) | (2.9) | | 15.1 to 36.5 | 57.3 to 80.1 |
| | Rhinorrhea | Day before | 25 | 30 | 12 | | | 36.2 | 79.7 |
| | (Runny | Day of Final | (36.2) | (43.5) | (17.4) | (2.9) | 69 | 25.0 to 48.7 | 68.3 to 88.4 |
| | Nose) | Visit | | | | | | | |
| | Nasal | Intermediate | 18 | 36 | 14 | 1 | 69 | 26.1 | 78.3 |
| | Blockage | Day | (26.1) | (52.2) | (20.3) | (1.4) | | 16.3 to 38.1 | 66.7 to 87.3 |
| | (Stuffy | Day before | 32 | 29 | 7 | 1 | 69 | 46.4 | 88.4 |
| | Nose) | Day of Final | (46.4) | (42.0) | (10.1) | (1.4) | | 34.3 to 58.8 | 78.4 to 94.9 |
| | | Visit | | | | | | | |

As shown in Table 16, for the sustained effects on paroxysmal sneezing and rhinorrhea, for the subject's impressions of sustained effects reached 24.6% for “Satisfied” and 69.6% for “Somewhat Satisfied” or better on day 3 of administration, and increased to 36.2% for “Satisfied” and 79.7% for “Somewhat Satisfied” or better on the day before the day of final visit. As for nasal blockage, the subject's impressions of sustained effects reached 26.1% for “Satisfied” and 78.3% for “Somewhat Satisfied” or better on day 3 of administration, and increased to 46.4% for “Satisfied” and 88.4% for “Somewhat Satisfied” or better on the day before the day of final visit.

### Therapeutic Effect Evaluation 3 (Evaluation of Final Overall Degree of Improvement by Investigators)

The investigators integrated the nasal cavity findings and nasal symptoms of each subject as well as subject's impressions, and determined the “final overall degree of improvement” in the present clinical trial on the following 5-point scale:
Markedly improved, 1; Moderately improved, 2; Somewhat improved, 3; Unchanged, 3: and Worsened, 5.

The final degree of improvement determined by the investigators was tabulated. Table 17 shows the results for 66 subjects who had their final visit on day 7 of administration and 2 subjects who had their final visit on day 6 of administration (total of 68 people) according to the clinical trial plan, and Table 18 shows the results for all subjects (69 people).

TABLE 17
Final overall degree of improvement (68 people who had
their final visit on day 7 or 6 of administration)

| | | | | | | Final Overall Improvement Rate (%) | |
|---|---|---|---|---|---|---|---|
| | | | | | | 95% Confidence Interval | |
| Number of Cases (Percentage (%)) | | | | | | “Moderately | “Somewhat |
| Markedly | Moderately | Somewhat | | | | Improved” | Improved” |
| Improved | Improved | Improved | Unchanged | Worsened | Total | or Better | or Better |
| 8 | 35 | 17 | 8 | 0 | 68 | 63.2 | 88.2 |
| (11.8) | (51.5) | (25.0) | (11.8) | (0.0) | | 50.7 to 74.6 | 78.1 to 94.8 |

TABLE 18
Final overall degree of improvement (day of final visit of 69 subjects)

| | | | | | | Final Overall Improvement Rate (%) | |
|---|---|---|---|---|---|---|---|
| | | | | | | 95% Confidence Interval | |
| Number of Cases (Percentage (%)) | | | | | | “Moderately | “Somewhat |
| Markedly | Moderately | Somewhat | | | | Improved” | Improved” |
| Improved | Improved | Improved | Unchanged | Worsened | Total | or Better | or Better |
| 8 | 35 | 18 | 8 | 0 | 69 | 62.3 | 88.4 |
| (11.6) | (50.7) | (26.1) | (11.6) | (0.0) | | 49.8 to 73.7 | 78.4 to 94.9 |

As described above, the final overall improvement rate is at least 62.3% for “Moderately Improved” or better and at least 88.2% for “Somewhat Improved” or better, in both tallies where the population is 68 and 69 people.

### Safety Confirmation:

The subjects in present clinical trial visited the hospital and had an examination by the investigators when they became aware of adverse events.

In the present clinical trial, 11 out of 69 subjects became aware of adverse events and had examination. The details, severity, and incidence of adverse events in 11 patients are as shown in Table 19 below. Here, the severity of adverse events was determined on the following three-point scale with reference to the “Standard for Classifying the Severity of Adverse Events of Medicines (Pharmaceutical Safety No. 80, Notification from director of Safety Division, Pharmaceutical Affairs Bureau, Ministry of Health and Welfare on Jun. 29, 1992 ‘On the Standard for Classifying the Severity of Side Effects of such as Medicines’”.
Mild: Degree that does not require treatment and is possible to continue administration of the investigational product
Moderate: Degree that requires treatment but is possible to continue administration of the investigational product
Severe: Degree that is necessary to stop the clinical trial

TABLE 19
Number of cases (number of events) and incidence rate by severity of adverse events

| System | | Mid | | | Moderate | | | Severe | | |
|---|---|---|---|---|---|---|---|---|---|---|
| Organ Class | | Number of | Number of | Incidence | Number of | Number of | Incidence | Number of | Number of | Incidence |
| (SOC) | Preferred Terts (PT) | Events | Cases | (%) | Events | Cases | (%) | Events | Cases | (%) |
| Total | | 11 | 11 | 15.9 | 0 | 0 | 0.0 | 0 | 0 | 0.0 |
| General disorders and | | 10 | 10 | 14.5 | 0 | 0 | 0.0 | 0 | 0 | 0.0 |
| administration site conditions | | | | | | | | | | |
| | Pain | 9 | 9 | 13.0 | 0 | 0 | 0.0 | 0 | 0 | 0.0 |
| | Irritation felt at site of | 1 | 1 | 1.4 | 0 | 0 | 0.0 | 0 | 0 | 0.0 |
| | Administration | | | | | | | | | |
| Infections and infestations | | 1 | 1 | 1.4 | 0 | 0 | 0.0 | 0 | 0 | 0.0 |
| | Nasopharyngitis | 1 | 1 | 1.4 | 0 | 0 | 0.0 | 0 | 0 | 0.0 |

As described above, all the above 11 adverse events were mild, that is, they did not require any treatment, so that it was possible to continue the administration of the investigational product. There were no moderate or severe adverse events. In addition, there were no cases in which the body temperature exceeded 37.5° C. during the clinical trial.

Table 20 below shows the details, severity, and incidence of the adverse events, among the above adverse events, for which the investigators judged that it was impossible to deny relationships to the present clinical trial (that is, events of s Adverse Drug Reaction).

TABLE 20
Number of cases (number of events) and incidence rate by severity of adverse drug reaction

| System | | | | | | | | | | |
|---|---|---|---|---|---|---|---|---|---|---|
| Organ | | Mild | | | Moderate | | | Severe | | |
| Class | Preferred | Number | Number | Incidence | Number | Number | Incidence | Number | Number | Incidence |
| (SOC) | Term (PT) | of Events | of Cases | (%) | of Events | of Cases | (%) | of Events | of Cases | (%) |
| Total | | 9 | 9 | 13.0 | 0 | 0 | 0.0 | 0 | 0 | 0.0 |
| General disorders and | | 9 | 9 | 13.0 | 0 | 0 | 0.0 | 0 | 0 | 0.0 |
| administration site | | | | | | | | | | |
| conditions | | | | | | | | | | |
| | Pain | 9 | 9 | 13.0 | 0 | 0 | 0.0 | 0 | 0 | 0.0 |

As shown in Table 20 there were 9 cases of adverse side reaction for which it was impossible to deny causal relationships to the present investigational product, but all of which were pain. They were all mild and resolved without treatment, and administration of the investigational product was continued until the end of the clinical trial. In addition, no clinically relevant abnormal changes were observed in laboratory test values or vital signs. There were no deaths or serious adverse events in the present clinical trial.

### Overall Safety:

On the day of final visit, the investigators comprehensively evaluated adverse events and determined the overall safety based on the following criteria.
Safe: No adverse drug reactions, etc., were observed.
Almost safe: As a general criterion, mild adverse drug reactions, etc., were observed.
Somewhat problematic: As a general criterion, moderate adverse drug reactions, etc., were observed.
Problematic: As a general criterion, severe adverse drug reactions, etc., were observed.

As a result, the overall safety was evaluated such that 87.0% (60/69 cases) was “safe” or better, and 100% (69/69 cases) was “almost safe” or better.

### Conclusions on the Clinical Trial

The present nasal composition containing 50 mg of oxymetazoline hydrochloride and 500 mg of chlorpheniramine maleate in 100 mL shows improved effects on all three major nasal symptoms of acute rhinitis or allergic rhinitis (sneezing, runny nose, and nasal blockage) when administered once or twice a day. In addition, it was confirmed that there was no problem with safety, as no adverse drug reaction that were particularly problematic were observed in this administration.

### Example 3 (Animal Experimentation 2)

The same animal test as in Example 1 was carried out with different concentrations of active ingredients.

Four-week-old male Hartley strain guinea pigs were subjected to quarantine and acclimatization, sensitization, induction, and selection in the same manner as in Example 1. The selected guinea pigs were grouped into E to M groups (10 animals in each group) by the stratified randomization method using the mean value of the symptom scores of each individual for 3 cycles as an index.

### Administration of Test Items:

The following test items E to M were administered to the guinea pigs of each group from the day after grouping.
E: Placebo
F: Composition containing 50 mg of chlorpheniramine maleate and 50 mg of oxymetazoline hydrochloride in 100 mL of the composition
G: Composition containing 200 mg of chlorpheniramine maleate and 50 mg of oxymetazoline hydrochloride in 100 mL of the solution
H: Composition containing 400 mg of chlorpheniramine maleate and 50 mg of oxymetazoline hydrochloride in 100 mL of the composition
I: Composition containing 600 mg of chlorpheniramine maleate and 50 mg of oxymetazoline hydrochloride in 100 mL of the composition
J: Composition containing 1000 mg of chlorpheniramine maleate and 50 mg of oxymetazoline hydrochloride in 100 mL of the composition
K: Composition containing 500 mg of chlorpheniramine maleate and 10 mg of oxymetazoline hydrochloride in 100 mL of the composition
L: Composition containing 500 mg of chlorpheniramine maleate and 45 mg of oxymetazoline hydrochloride in 100 mL of the composition
M: Composition containing 500 mg of chlorpheniramine maleate and 100 mg of oxymetazoline hydrochloride in 100 mL of the composition

All the above E to M contain the same amounts of the same additives as in Example 1.

The test items were nasally administered to the guinea pigs in the same manner as in Example 1, the symptoms were observed and evaluated, and the symptom score of each animal was calculated. The results were as shown in Table 21.

TABLE 21

| | | Concentration of Active Ingredient | | | | |
|---|---|---|---|---|---|---|
| | Amount of | | | | | Mean Value and |
| | Solution | Chlorpheniramine | | Oxymetazoline | | Standard Error of |
| Group | Administered | Maleate | | Hydrochloride | | Symptom Scores |
| E | 200 μL | — | | — | | 8.2 ± 0.2 |
| F | 200 μL | 0.05% | (50 mg)* | 0.05% | (50 mg)* | 6.5 ± 0.5 |
| G | 200 μL | 0.2% | (200 mg)* | 0.05% | (50 mg)* | 4.8 ± 0.7 |
| H | 200 μL | 0.4% | (400 mg)* | 0.05% | (50 mg)* | 4.0 ± 0.7 |
| I | 200 μL | 0.6% | (600 mg)* | 0.05% | (50 mg)* | 3.0 ± 0.6 |
| J | 200 μL | 1.0% | (1000 mg)* | 0.05% | (50 mg)* | 3.4 ± 0.7 |
| K | 200 μL | 0.5% | (500 mg)* | 0.01% | (10 mg)* | 5.0 ± 0.6 |
| L | 200 μL | 0.5% | (500 mg)* | 0.045% | (45 mg)* | 3.6 ± 0.7 |
| M | 200 μL | 0.5% | (500 mg)* | 0.1% | (100 mg)* | 2.7 ± 0.8 |

*Content of each component per 100 mL of test item

E above is a placebo, and F above contains the active ingredients chlorpheniramine maleate and oxymetazoline hydrochloride in the same amounts as Respibien. G to M above are examples of nasal compositions according to the present invention. From the above, it is apparent to those skilled in the art that the nasal composition according to the present invention is superior to Respibien in its efficacy.

What is claimed is:

1. A method of treating acute rhinitis and/or allergic rhinitis; comprising a step of spraying a composition to a nostril of a patient with acute rhinitis and/or allergic rhinitis once or twice a day, at intervals of 10 hours or longer, for a period of up to 7 days;
wherein the composition comprises (a) chlorpheniramine or its pharmaceutically acceptable salt and (b) oxymetazoline or its pharmaceutically acceptable salt; and
wherein the amount of (a) in terms of chlorpheniramine maleate is 200 to 1000 mg per 100 mL of the composition and the amount of (b) in terms of oxymetazoline hydrochloride is 10 to 100 mg per 100 mL of the composition.

2. The method according to claim 1, wherein the amount of (a) is 200 to 600 mg per 100 mL of the composition.

3. The method according to claim 2, wherein the amount of (b) is 45 to 100 mg per 100 mL of the composition.

4. The method according to claim 3, wherein the amount of (a) is 400 to 600 mg per 100 mL of the composition.

5. The method according to claim 4, wherein the amount of (b) is 45 to 50 mg per 100 mL of the composition.

6. The method according to claim 5, wherein the amount of (a) is 400 to 500 mg per 100 ml of the composition.

7. The method according to claim 6, wherein the amount of (a) is 500 mg per 100 mL of the composition.

8. The method according to claim 6, wherein the amount of (b) is 50 mg per 100 mL of the composition.

9. The method according to claim 1, wherein (a) is chlorpheniramine maleate, and (b) is oxymetazoline hydrochloride.

10. The method according to claim 1, wherein the patient has developed symptoms of acute rhinitis and/or allergic rhinitis.
